(19) United States
(12) Patent Application Publication
(10) Pub. No.: US 2026/0034196 A1
(43) Pub. Date: Feb. 5, 2026

(54) THERAPEUTIC PRODUCT MIXING METHODS

(71) Applicant: AMGEN INC., Thousand Oaks, CA (US)

(72) Inventors: Kevin R. Ryan, Thousand Oaks, CA (US); Iman Mirzaee Kakhki, Thousand Oaks, CA (US); Venkata Ramana Ramaka, Thousand Oaks, CA (US); Arturo G. Soto, Thousand Oaks, CA (US); Kathrine Bradford Atkins, Thousand Oaks, CA (US)

(21) Appl. No.: 19/100,765

(22) PCT Filed: Aug. 11, 2023
(86) PCT No.: PCT/US2023/030029

§ 371 Date: Feb. 3, 2025

Related U.S. Application Data
(60) Provisional application No. 63/397,611, filed on Aug. 12, 2022.

(51) Int. Cl. A61K 38/17; B01D 61/14; B01F 23/43; B01F 23/80; B01F 27/113; B01F 27/61; B01F 27/71; B01F 35/50; B01F 101/22

(52) U.S. Cl. CPC A61K 38/1793; B01D 61/145; B01F 23/43; B01F 23/808; B01F 27/113; B01F 27/61; B01F 27/71; B01F 35/50; B01D 2315/16; B01F 2101/22; B01F 2215/0431; B01F 2215/0459; B01F 2215/0481; B01F 2215/0495

(57) ABSTRACT

Methods for mixing fluids including etanercept include providing a tank having an impeller with a 10 inch blade diameter disposed therein, filling a tank with a liquid composition comprising etanercept and one or more buffers to a level within the tank to cover the impeller, and operating the impeller to mix the liquid composition to a homogeneous state while maintaining a maximum shear stress imparted on the mixture of fluids at 125,000 s⁻¹ or below.

### CROSS-REFERENCE TO RELATED APPLICATIONS

Priority is claimed to U.S. Provisional Patent Application No. 63/397,611, filed Aug. 12, 2022, the entire contents of which are incorporation by reference herein.

### FIELD OF THE DISCLOSURE

The present disclosure relates to processes for making therapeutic products and, more particularly, mixing processes for making therapeutic products.

### BACKGROUND

Therapeutic products containing proteins are susceptible to shear stresses, which can damage the protein structure of the product. This susceptibility must be accounted for in the ultrafiltration/diafiltration process utilized to concentrate and produce a final product. As understood, the ultrafiltration/diafiltration process includes stages where the product is mixed with buffers within a tank. Commercial processes can utilize an impeller that extends within the tank to achieve a homogeneous mixture of the fluids. Many products are less susceptible to shear stresses and, as a result, larger impellers, such as those with a 10 inch blade diameter, can be used without concern for damaging the end product. When the product line is switched over to more susceptible products, however, changing the size of the impeller results in significant downtime for a user to enter the tank to change the impeller, clean the tank, test the tank for cleanliness, and so forth. Additionally, protocols for user entry can be avoided, as well as improving the safety related to the manufacturing process.

### SUMMARY

In accordance with one implementation, a method for mixing a liquid composition is described that includes providing a tank having an impeller with a 10 inch blade diameter disposed therein, filling a tank with a liquid composition comprising etanercept and one or more buffers to a level within the tank to cover the impeller, and operating the impeller to mix the liquid composition to a homogeneous state while maintaining a maximum shear stress imparted on the mixture of fluids at 125,000 s⁻¹ or below.

In some examples, the maximum shear stress imparted on the liquid composition can be less than 55,000 s⁻¹ or less than 30,000 s⁻¹.

In some examples, the method can include performing a buffer exchange pursuant to an ultrafiltration/diafiltration process and/or filling the tank with the liquid composition can include filling the tank with a concentrated therapeutic product including etanercept and a final buffer.

In some examples, operating the impeller can produce a power/flow number ratio less than 1 or less than 0.7.

In some examples, operating the impeller can include driving rotation of the impeller at less than 250 rpm. In further examples, operating the impeller can include maintaining a volume average turbulence kinetic energy (TKE) of at least 0.005 m²/s² and/or having a turbulent dissipation rate (TDR) of at least 0.035 m²/s³.

In any of the above examples, the method can include one or more of the following aspects: the method can include operating the impeller to maintain an average shear rate of less than 8 s⁻¹ throughout the liquid composition; the tank can be a tulip-shaped tank with a bottom stem portion and a top bulb portion and operating the impeller can include driving the impeller via a connection through the bottom stem portion of the tank; operating the impeller can include driving rotation of the impeller to pull the liquid composition upward from a bottom of the tank; the tank can have a volume of 210 L or less or 140 L or less; the impeller can include three blades having a tapering profile; and/or the liquid composition in the homogenous state can have a viscosity between about 3 cp and about 8 cp.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings are necessarily to scale.

FIG. 1 is a diagrammatic view of a tank for an ultrafiltration/diafiltration process.

FIG. 2 is a perspective view of an impeller for mixing fluids in the tank of FIG. 1.

FIG. 3 is a diagrammatic view of an ultrafiltration/diafiltration system.

FIGS. 4A-4D illustrate X-plane velocity contours for four example cases generated by computational fluid dynamics modeling of rotating impellers mixing fluids within respective tanks.

FIGS. 5A-5D illustrate horizontal plane velocity contours for the four example cases of FIGS. 4A-4D.

FIGS. 6A-6D illustrate X-plane velocity vectors for the four example cases of FIGS. 4A-4D.

FIGS. 7A-7D illustrate horizontal plane velocity vectors for the four example cases of FIGS. 4A-4D.

FIGS. 8A-8D illustrate X-plane shear rate contours for the four example cases of FIGS. 4A-4D.

FIGS. 9A-9D illustrate X-plane spatial distribution contours of mean age for the four example cases of FIGS. 4A-4D.

FIGS. 10A-10D illustrate horizontal plane spatial distribution contours of mean age for the four example cases of FIGS. 4A-4D.

FIGS. 11A and 11B are mean age histogram plots for the four example cases of FIGS. 4A-4D.

FIGS. 12A and 12B illustrate X-plane velocity contours for two example cases generated by computational fluid dynamics modeling of rotating impellers mixing fluids within respective tanks.

FIGS. 13A and 13B illustrate horizontal plane velocity contours for the two example cases of FIGS. 12A and 12B.

FIGS. 14A and 14B illustrate X-plane velocity vectors for the two example cases of FIGS. 12A and 12B.

FIGS. 15A and 15B illustrate horizontal plane velocity vectors for the two example cases of FIGS. 12A and 12B.

FIGS. 16A and 16B illustrate X-plane shear rate contours for the two example cases of FIGS. 12A and 12B.

FIGS. 17A and 17B illustrate X-plane spatial distribution contours of mean age for the two example cases of FIGS. 12A and 12B.

FIGS. 18A and 18B illustrate horizontal plane spatial distribution contours of mean age for the two example cases of FIGS. 12A and 12B.

FIGS. 19A and 19B are mean age histogram plots for the two example cases of FIGS. 12A and 12B.

### DETAILED DESCRIPTION

The present disclosure relates to a method for mixing fluids, such as a liquid composition comprising a shear stress susceptible protein, such as etanercept, with an impeller having a 10 inch blade diameter. The fluids are fed into a tank where the impeller is mounted. The method can advantageously be part of an ultrafiltration/diafiltration process, e.g., pursuant to a buffer exchange or final buffer addition. The impeller is driven at a desired rotation per minute (rpm) to maintain a maximum shear stress below a predetermined threshold known to produce a satisfactory product. The methods described herein advantageously produce a better mixture of the fluids, while operating the impeller at a lower rpm and using less power.

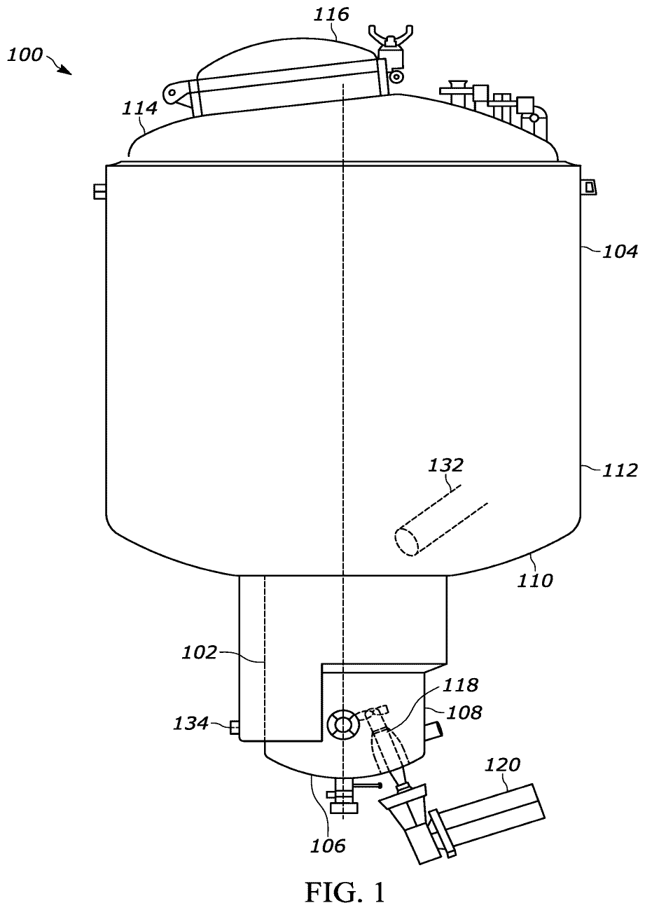

An example tank 100 is shown in FIG. 1. The tank 100 has a tulip configuration with a lower stem portion 102 and an enlarged, upper bulb portion 104. The stem portion 102 has a closed bottom wall 106 and an annular sidewall 108 having a first diameter. The bulb portion 104 has a ring-shaped bottom wall 110 and an annular sidewall 112 having a second diameter, which is larger than the first diameter. As shown, the bulb bottom wall 110 extends around a top edge of the stem sidewall 108. The bulb bottom wall 110 can have a downwardly angled or curved, frusto-conical configuration to direct fluid flow into the stem portion 102. A top wall 114 of the bulb portion 104 can be a removable cover or can include a separate protruding removable cover 116, as shown. An impeller 118 is mounted within the tank 100 and configured to be driven to mix fluids in the tank 100. For example, the impeller 118 can be operably coupled to a drive 120, such as a magnetic drive, to drive rotation thereof. The tank 100 can include an inlet 132 opening through the bulb portion 104 thereof. For example, the inlet 132 can introduce fluids into the tank 100 just above the transition between the bulb portion 104 and the stem portion 102. The tank 100 can also include an outlet 134 in the stem portion 102 to allow fluids to drain from the tank 100. For example, the outlet 134 can be through the sidewall 108 of the stem portion 102 adjacent to the bottom wall 106 thereof.

In one example, the stem portion 102 can have a diameter of 18 inches and the bulb portion 104 can have a diameter of 48 inches. With this configuration, and the shape of the bulb portion bottom wall 110, a 140 L liquid level has a height of 30.24 inches in the tank 100 and a 210 L liquid level has a height of 33.15 inches in the tank 100.

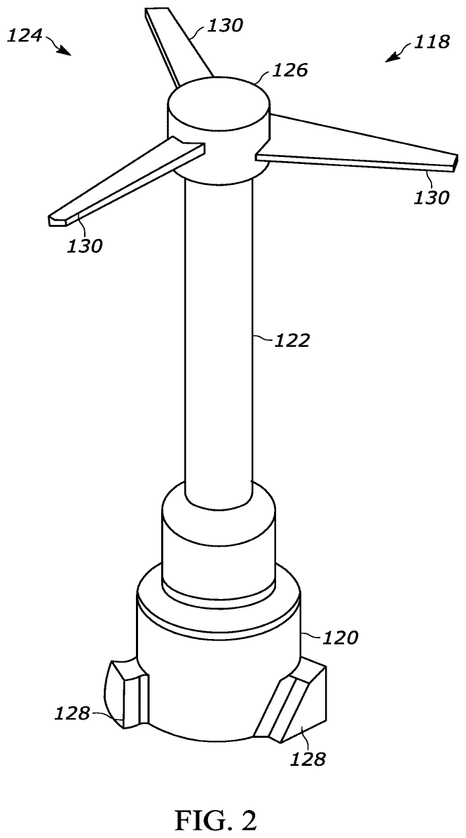

Details of an example impeller 118 are shown in FIG. 2. The impeller 118 for the system includes a base 120, a central shaft 122, and a blade array 124 extending radially outwardly from a distal end 126 of the shaft 122. As shown, the base 120 can have a cylindrical stepped configuration with one or more angled tabs 128 extending outwardly therefrom to disrupt fluid flow around the base 120. In the illustrated example, the impeller 118 includes three blades 130 in the blade array 124. Each blade 130 has a tapering profile and an angled configuration relative to a longitudinal axis of the impeller 118. In the illustrated form, the blades 130 are angled downward in a clockwise direction and the impeller 118 is configured to be driven to pull fluid upwardly within the tank 100. Further, as discussed above, the distal ends of the blades 130 rotate in a circle having a 10 inch diameter.

The impeller 118 is mounted within the stem portion 102 of the tank 100 to extend away from the stem bottom wall 106. As shown, the impeller 118 can be mounted in an off-center location with respect to the bottom wall 106 and can extend an angle with respect to a vertical axis of the tank 100, such that the impeller 118 extends across an interior of the stem portion 102.

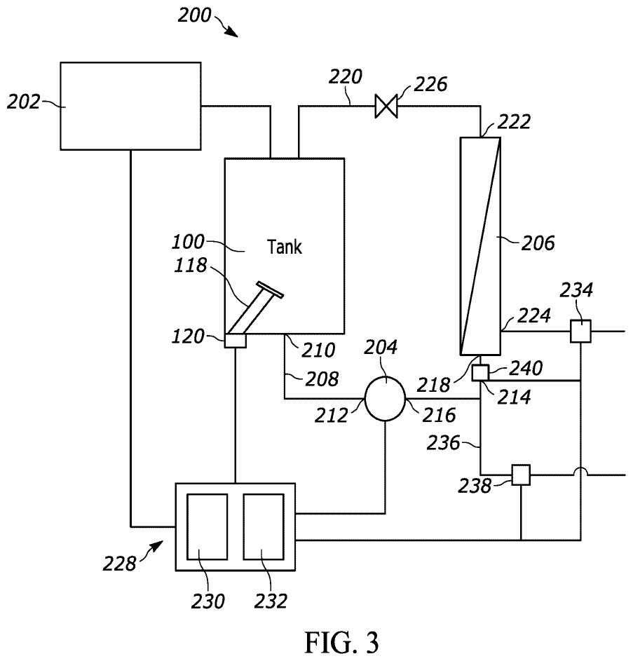

The tank 100 and impeller 118 can be used within an ultrafiltration/diafiltration (UFDF) system 200, as shown in FIG. 3. For example, the tank 100 can be used pursuant to a buffer exchange, product concentration, final buffer addition, etc. The tank 100 is fluidly connected to an upstream unit or units 202. The upstream unit or units 202 can include a chromatography processing unit, a viral filtration processing unit, a storage tank, and so forth. The system 200 further includes a feed pump 204 and a filter 206, such as a tangential flow filter (TFF). A line 208 is connected to an outlet 210 of the tank 100 and an inlet 212 of the pump 204. A further line 214 is connected to an outlet 216 of the pump 204 and an inlet 218 of the filter 206. A retentate return line 220 is connected to an outlet 222 of the filter 206 and the tank 100. Permeate exits the system 200 at a permeate outlet 224 of the filter 206. A backpressure valve 226 can be provided along the retentate return line 220. A sensor 234 can be operably coupled to the permeate line to determine the permeate flow. As shown, a tank outlet line 236 can be fluidly coupled to the outlet 216 of the feed pump 204 to thereby direct fluid flow from the tank 100 to other components of the system 200. The lines 214, 236 coupled to the outlet 216 of the feed pump 204 can each include valves 238, 240 to restrict fluid flow therethrough.

In some implementations, the system 200 can include a control system 228. The control system 228 may be operably coupled to the upstream unit or units 202, the feed pump 204, the impeller drive 120 to selectively drive rotation of the impeller 118, the sensor 234, and the valves 238, 240. The control system 228 may be configured or adapted to control the components to carry out any desired UFDF processing steps. According to certain examples, the control system 228 may include one or more processors 230 and memory 232, the memory 232 coupled to the one or more processors 230. The one or more processors 230 may be programmed to control the upstream unit or units 202 and the pump 206. The instructions executed by the one or more processors 230 may be stored on the memory 232, which memory 232 may comprise tangible, non-transitory computer-readable media or storage media, such as read-only memory (ROM) or random access memory (RAM) in a variety of forms (e.g., hard disk, optical/magnetic media, etc.).

It has been shown utilizing Computational Fluid Dynamics (CFD) modeling, as discussed in more detail below, that not only can a 10 inch impeller be used instead of a 4 inch impeller for mixing a liquid composition comprising etanercept and other similar shear stress susceptible products, but that a 10 inch impeller can provide a more homogeneous mixture than the 4 inch impeller, reduce local recirculation zones within the tank 100, and utilize less power to create a homogeneous mixture. As shown below, it was found that the 10 inch impeller can be operated to create a homogeneous mixture of the fluids in the tank 100, while maintaining a maximum shear stress imparted on the mixture of fluids at 125,000 s⁻¹ or below. In further examples, the maximum shear stress imparted on the mixture can be less than 55,000 s⁻¹ or less than 30,000 s⁻¹. Further, it was found that the 10 inch impeller can be operated to maintain an average shear rate of less than 8 s⁻¹ throughout the mixture.

The single-phase CFD models described below provided an understanding of the flow field and mean age distribution characteristics of the tank 100 for the manufacture of product composition comprising etanercept. Results from the various models were compared between 10 and 4 inch impeller configurations. For the below examples, ANSYS/Fluent Software version 19.2 was used to perform the CFD modeling and simulations. An ANSYS Design-Modeler (DM) meshing tool was used to generate hex dominant grids consisting of tetrahedrals. The maximum cell skewness in the generated meshes was below the level recommended by ANSYS.

An ANSYS Fluent finite volume solver was used for the CFD simulations. The meshed geometries were imported into ANSYS Fluent to compute single phase flow field using the Navier Stokes equations coupled with the ANSYS Fluent turbulence model. A moving reference frame (MRF) model was used to account for the impeller motion. Boundary conditions used in the stirred single-phase model for a flow-through system are summarized as follows: 1. No-slip boundary with a standard wall function on all walls; 2. Symmetry planes on liquid surfaces; 3. Mass flow inlet at fluid inlets; and 4. Pressure outlet at fluid outlet.

The model input parameters used for single-phase CFD models to predict velocity fields and mean age distribution are summarized in Table 1.

TABLE 1
Model Inputs for Flow-Through System

| Parameters | Set 1 | Set 2 |
|---|---|---|
| Working Volume (L) | 140 | 210 |
| Agitation Rate (rpm) | 207 and 345 | 207 and 345 |
| Liquid Density (kg/m³) | 1074 | 1028 |
| Liquid Viscosity (cp) | 7.21 | 3.23 |

In a first approach, performed in modeling cycle 1 (MC1), two sets of cases were tested with similar settings for a 4 inch impeller and a 10 inch impeller. The cases of MC1 provided a better understanding of the mixing performance within the tank 100 in an example UF/DF process by analyzing the mean age distribution in the tank 100 for the both the 4 inch and 10 inch impeller configurations with the same agitation rates and same working volumes.

In a second set, performed in modeling cycle 2 (MC2), one set of cases was tested to study the mean age distribution at reduced agitation rates for the 10 inch impeller relative to the 4 inch impeller. The objected of MC2 was to decrease the average shear rate in the tank for the 10 inch impeller while keeping the mixing performance at satisfactory levels relative to the 4 inch impeller operating at a higher agitation rate.

For both MC1 and MC2, the following assumptions were made when running the CFD models and/or analyzing the results: 1. Material Properties (density and viscosity) were constant; 2. Liquid was assumed to be incompressible and Newtonian; 3. Headspace effects were negligible; 4. Geometry & Meshes are an approximate representation of the real system; 5. Flow from the F1A will not cause any splashing and the flux fluid enters the top surface of the bulk fluid from the circular inlet region on the side; and 6. A moving reference frame (MRF) was used to account for the impeller motion.

Modeling cycle 1 (MC1) compared the mixing performance by analyzing the mean age distribution in the tank for the 4 inch impeller and the 10 inch impeller configurations at the same agitation rates (207 and 345 rpm) and same working volumes (140 L and 210 L). The MC1 model concluded that, at the same agitation rates and the same working volumes, the 10 inch impeller has better mixing performance compared to the 4 inch impeller. Also, the MC1 model concluded that the 10 inch impeller has a higher shear rate compared to the 4 inch impeller due to a higher tip speed at 345 RPM.

Table 2 provides additional input parameters used for MC1 cases.

TABLE 2
Model Inputs for MC1 cases

| | Working | Impeller | Impeller | Flow Rates (lpm) | | | | |
|---|---|---|---|---|---|---|---|---|
| | Volume | Speed | Diameter | Feed | Return | Flux | Viscosity | Density |
| Case | (L) | (rpm) | (inches) | D2 | F2 | F1 | (cP) | (kg/m³) |
| 1 | 140 | 207 | 10 | 96 | 88 | 8 | 7.21 | 1074 |
| 2 | 140 | 207 | 4 | 96 | 88 | 8 | 7.21 | 1074 |
| 3 | 210 | 345 | 10 | 120 | 110 | 10 | 3.23 | 1028 |
| 4 | 210 | 345 | 4 | 120 | 110 | 10 | 3.23 | 1028 |

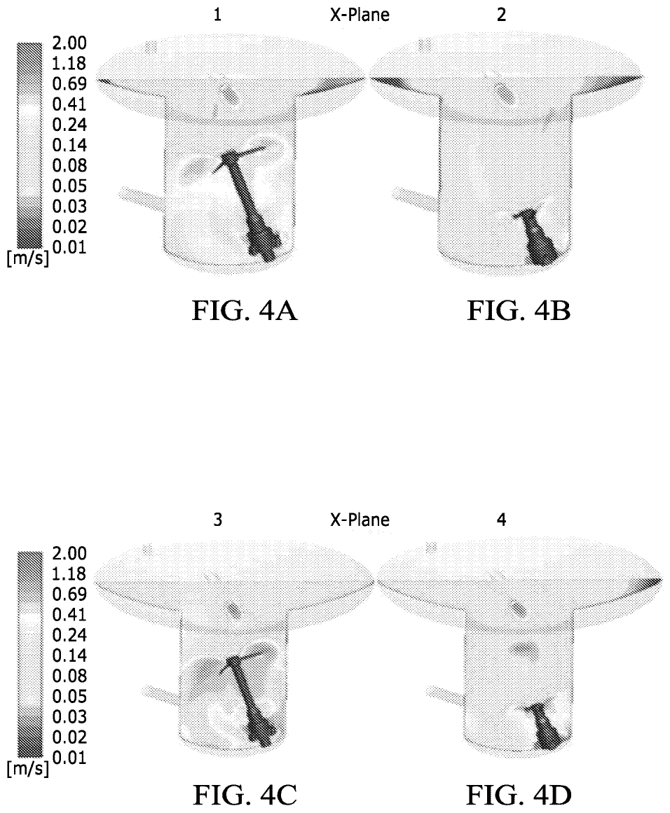

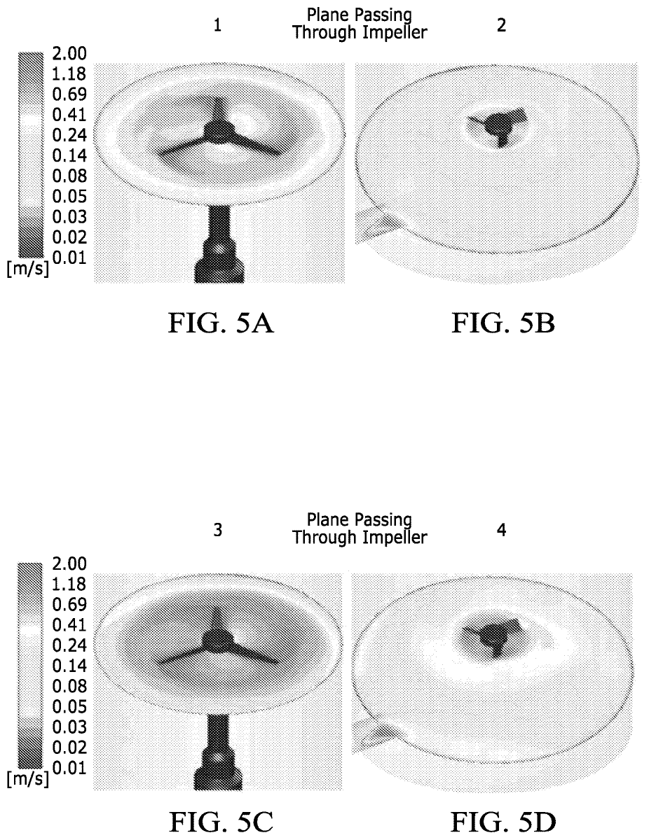

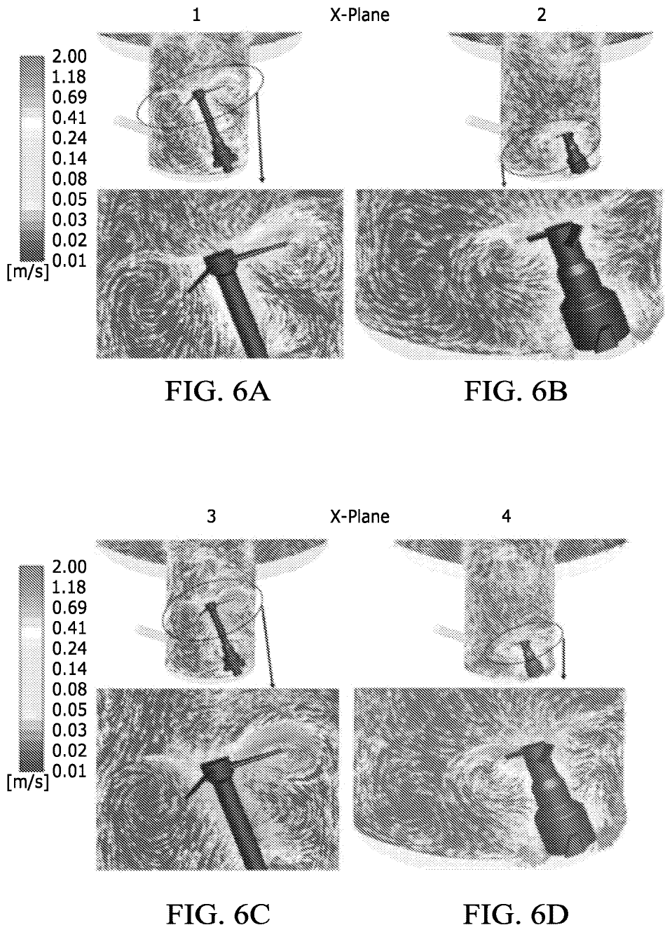

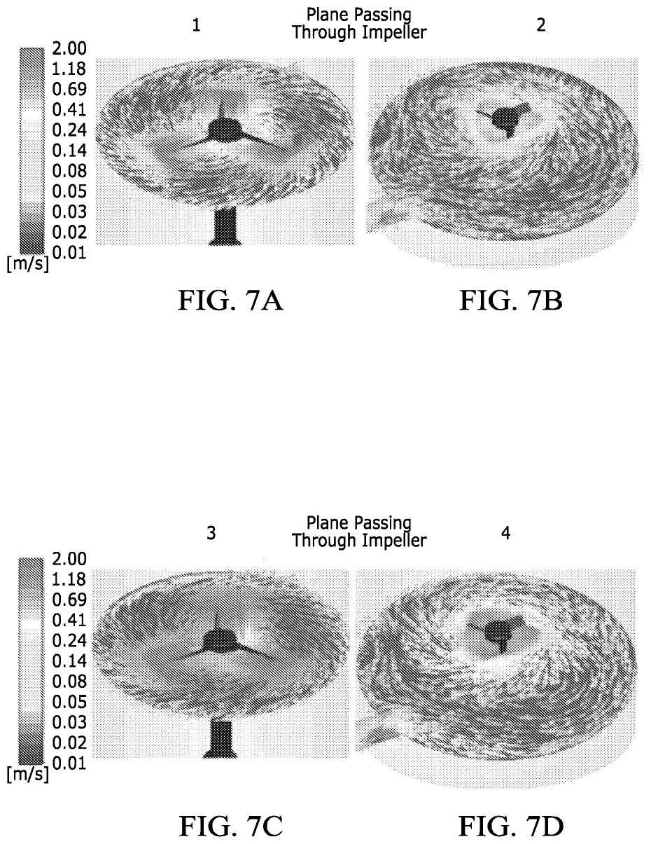

Steady state flow simulations were carried out to obtain the velocity contours and vectors, shear rate contours, and mean age contours in the vertical plane (X-plane) and horizontal plane passing through the impeller. FIGS. 4A-4D show velocity contours on the X-plane and FIGS. 5A-5D show velocity contours on the horizontal plane passing through the impeller for the four cases of MC1. A comparison of the 10 inch and 4 inch impellers at the same agitation rate and working volumes shown in FIGS. 4A, 4B and FIG. 4C, 4D, illustrates that the 10 inch impeller showed higher velocities than the 4 inch impeller due to the relatively larger blades 130. The 4 inch impeller cases have low velocity zones in stem portion 102 of the tank 100 and the bulb portion 104 of the tank 100, which provides evidence that the 10 inch impeller mixes the fluids in the tank 100 better than the 4 inch impeller. Velocity vectors on the X-plane for each of the cases in MC1 are shown in FIGS. 6A-6D. The velocity vectors illustrate recirculation zones on either side of the impellers. Velocity vectors on the horizontal plane passing through the impeller for each of the four cases of MC1 are shown in FIGS. 7A-7D. The velocity vectors in illustrate a clockwise motion of fluid in the tank 100.

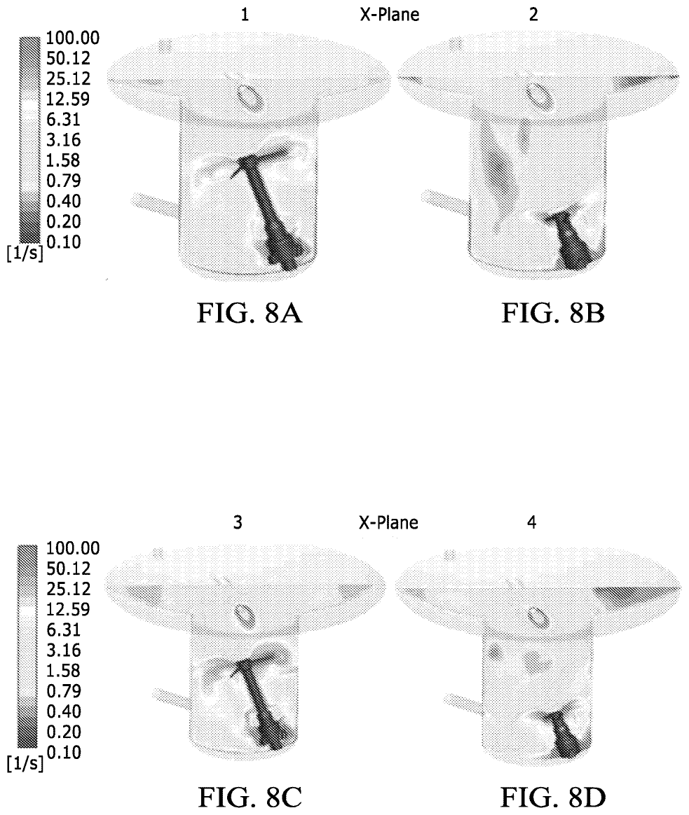

Shear rate contours on the X-plane for the four cases of MC1 are illustrated in FIGS. 8A-8D. As shown, higher shear rates are observed for the 10 inch impeller (FIGS. 8A and 8C) than the 4 inch impeller (FIGS. 8B and 8D), which is due to the increased impeller tip speed for the 10 inch impeller at the same agitation rate. Further, low shear zones are observed at both the stem portion 102 and the bulb portion 104 of the tank 100 in the 4 inch impeller (FIGS. 8B and 8D). Another feature to note is that the higher agitation rate cases shown in FIGS. 8C and 8D have higher maximum shear rates than the high viscosity cases shown in FIGS. 8A and 8B, but also have lower average shear rates.

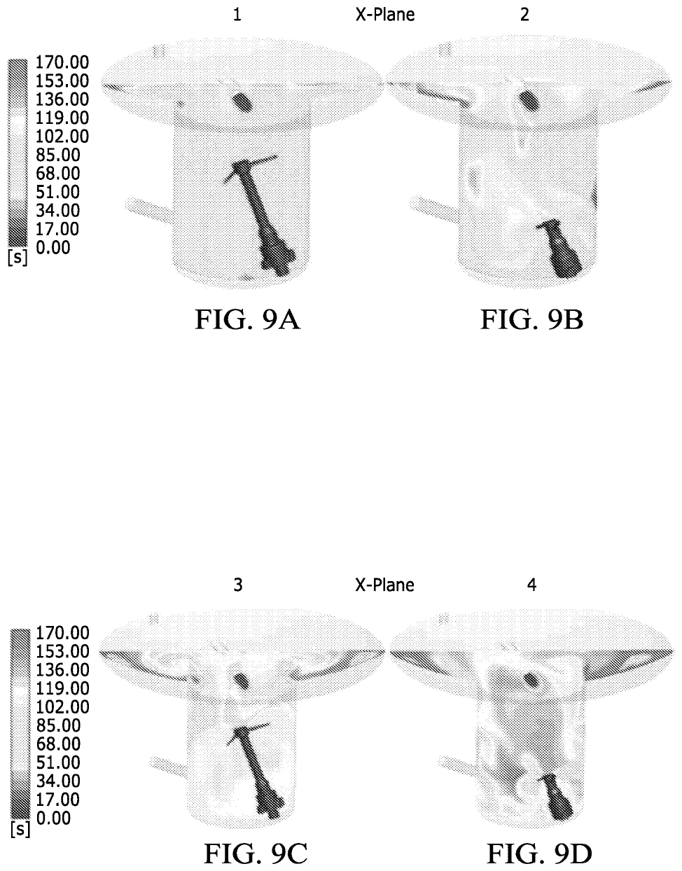

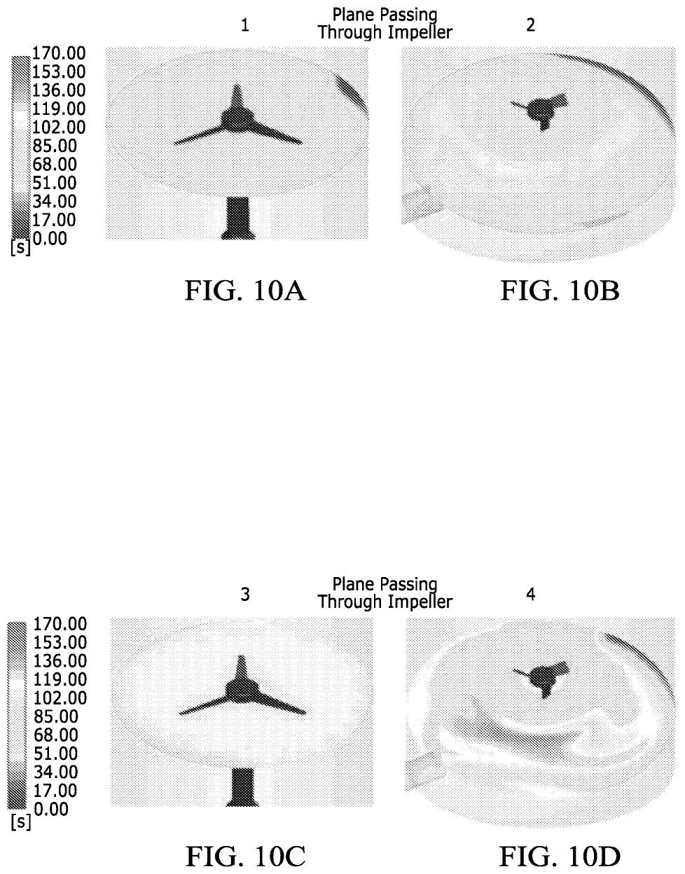

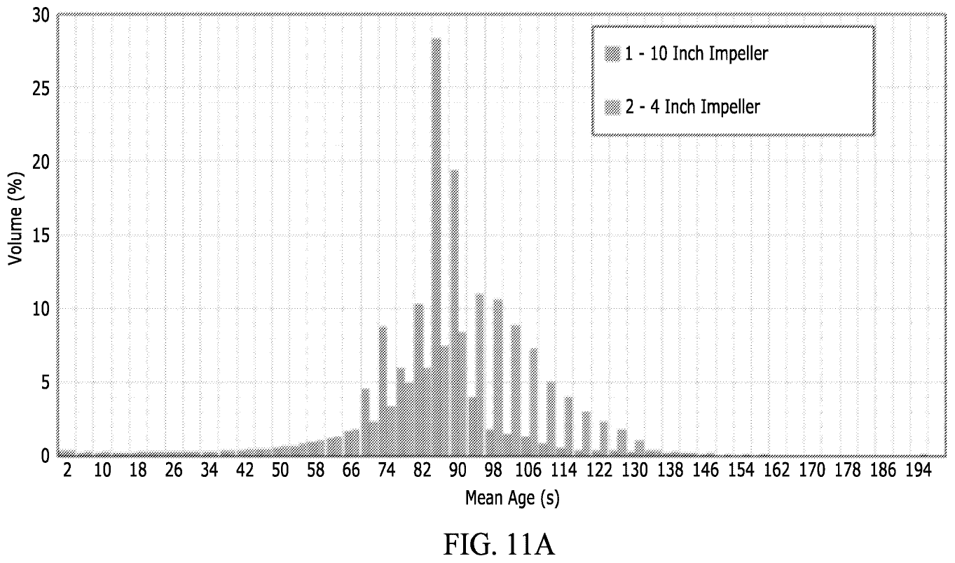

FIGS. 9A-9D show the spatial distribution (contours) of mean age on the X-plane and FIGS. 10A-10D shown the spatial distribution (contours) of mean age on the horizontal plane passing through the impeller for the four cases of MC1. For both agitation rates and working volumes, the 10-inch impeller results in a more homogeneous mean age distribution as compared to the 4 inch impeller. A homogeneous mean age distribution represents better mixing by the 10 inch impeller relative to the 4 inch impeller. Additionally, the mean age contours show that the longest time spent by fluid is in a region near the top of the bulb portion 104 of the tank 100, while the shortest time spent by fluid is near an inlet 132 to the tank 100 (see, FIG. 1).

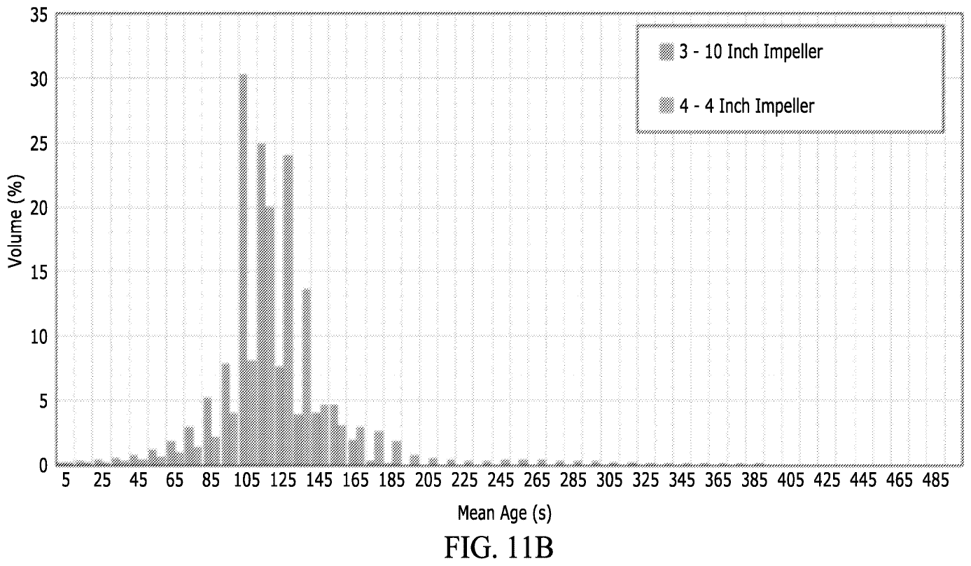

FIGS. 11A and 11B illustrate the mean age histogram plots for the cases in MC1. Cases 1 and 2 are shown in FIG. 11A and cases 3 and 4 are shown in FIG. 11B. A wider range of mean age values are observed in Cases 2 and 4 for the 4 inch impeller than those observed in Cases 1 and 3 for the 10 inch impeller. For the 10 inch impeller cases, ˜70% of the tank volume has a mean age value close to the retention time (87 s). In summary, each of these observations show a better mixing performance in the 10 inch impeller cases compared to the 4 inch impeller cases.

The area weighted average of mean age is taken at the tank exit (a_e) and volume weighted average of mean age is calculated for whole tank volume, excluding inlet pipe volumes, (a_v) are shown in Table 3. The standard deviation of the mean age average at the tank exit and tank volume is traditionally used for quantitative comparison of the mixing performance in flow through systems. A theoretical value of 1 for standard deviation is an indication of perfect mixing. The standard deviation of mean age for the 10 inch impeller on average is closer to 1 when compared to the 4 inch impeller, which provides another confirmation of the better mixing performance of the 10 inch impeller.

TABLE 3
Standard deviation of mean age for MC1 cases
Flow studies at nominal conditions

| | | | | | | | a_e | a_v | | | |
|---|---|---|---|---|---|---|---|---|---|---|---|
| | Working | Impeller | | Flow Rates (lpm) | | | Area | Volume | tau | | Sigma_e |
| | Volume | Speed | Diameter | Feed | Return | Flux | Weighted | Weighted | Retention | Sigma_e² | Standard |
| Case | (L) | (rpm) | (inches) | D2 | F2 | F1 | Ave. | Ave. | time (s) | Variance | Deviation |
| 1 | 140 | 207 | 10 | 96 | 88 | 8 | 87.71 | 85.25 | 87.5 | 0.94 | 0.972 |
| 2 | 140 | 207 | 4 | 96 | 88 | 8 | 85.5 | 94.93 | 87.5 | 1.22 | 1.105 |
| 3 | 210 | 345 | 10 | 120 | 110 | 10 | 104.41 | 110.26 | 105 | 1.11 | 1.055 |
| 4 | 210 | 345 | 4 | 120 | 110 | 10 | 106.48 | 138.62 | 105 | 1.6 | 1.266 |

A steady state solution was used to perform the mean age analysis. Standard deviation of mean age was estimated and flow behavior was characterized based on: standard deviation=1 indicates perfect mixing, standard deviation of greater than 1 indicates short-circuiting, and standard device less than 1 indicates plug-flow behavior.

A summary of the performance parameters for the cases in MC1 is shown in Table 4. With the same agitation rate and working volume, the 10 inch impeller had more power consumption than the 4 inch impeller. Further, the 10 inch impeller also had a higher average velocity and shear rate than the 4 inch impeller.

TABLE 4
Performance Summary for MC1 Cases

| Parameters | Units | Case 1 | Case 2 | Case 3 | Case 4 |
|---|---|---|---|---|---|
| Impeller Tip Speed | m/s | 2.76 | 1.101 | 4.6 | 1.84 |
| Maximum Shear Rate | s⁻¹ | 23218.5 | 5102.6 | 54842.6 | 8212.1 |
| Impeller Power Consumption | kW | 0.0102 | 0.0023 | 0.0448 | 0.0102 |
| Impeller Pumping Capacity | m³/s | 0.022 | 0.0045 | 0.034 | 0.0077 |
| Impeller Power Number | — | 0.22 | 4.8 | 0.22 | 4.84 |
| Impeller Flow Number | — | 0.38 | 1.24 | 0.36 | 1.27 |

Average Quantities

| | | | | | |
|---|---|---|---|---|---|
| Total Power Consumption | kW | 0.0102 | 0.0023 | 0.0448 | 0.0102 |
| Power Per-Unit Volume | kW/m³ | 0.073 | 0.016 | 0.213 | 0.049 |
| Average Strain Rate | 1/s | 7.49 | 4.41 | 7.14 | 4.22 |
| Volume Average Velocity | m/s | 0.211 | 0.101 | 0.271 | 0.127 |
| Volume Average TKE | m²/s² | 0.0076 | 0.0031 | 0.0141 | 0.0057 |
| Volume Average TDR | m²/s³ | 0.050 | 0.015 | 0.149 | 0.043 |
| Micromixing time calculated based | s | 0.197 | 0.355 | 0.078 | 0.145 |
| on Average TDR (epsilon) | | | | | |

Modeling cycle 2 (MC2) performed a shear rate analysis by comparing the mixing performance and analyzing the mean age distribution in the tank for a reduced agitation rate (207 rpm) for the 10 inch impeller in a 210 L working volume and a higher agitation rate (345 rpm) for the 4 inch impeller in a 140 L working volume. In addition to the comparison between the two cases, these simulations can also be used to compare the performance of the tanks with the four MC1 cases.

Table 5 provides input parameters used for the MC2 cases.

TABLE 5
Model Inputs for MC2 cases

| | Working | Impeller | Impeller | Flow Rates (lpm) | | | | |
|---|---|---|---|---|---|---|---|---|
| | Volume | Speed | Diameter | Feed | Return | Flux | Viscosity | Density |
| Case | (L) | (rpm) | (inches) | D2 | F2 | F1 | (cP) | (kg/m³) |
| 5 | 140 | 345 | 4 | 96 | 88 | 8 | 7.21 | 1074 |
| 6 | 210 | 207 | 10 | 120 | 110 | 10 | 3.23 | 1028 |

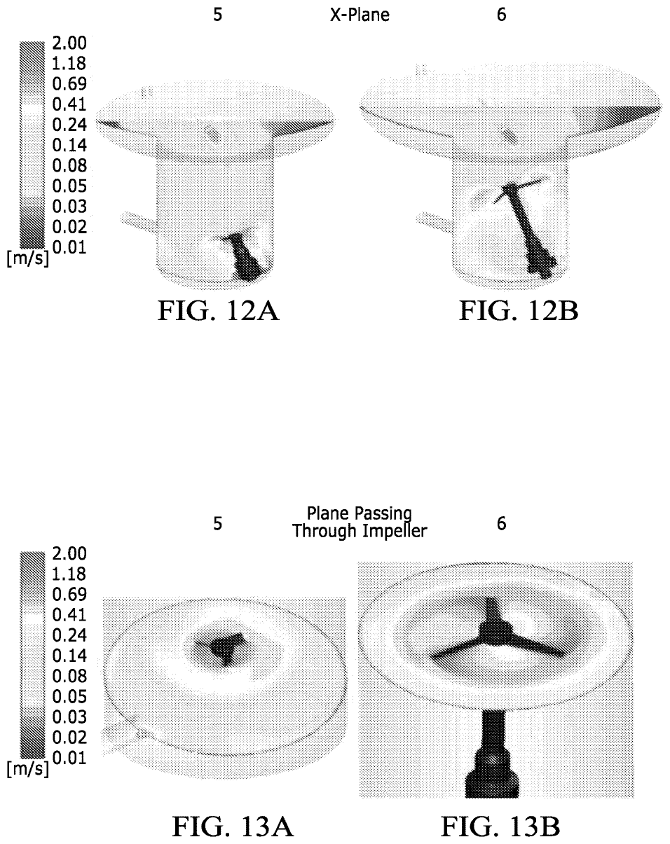

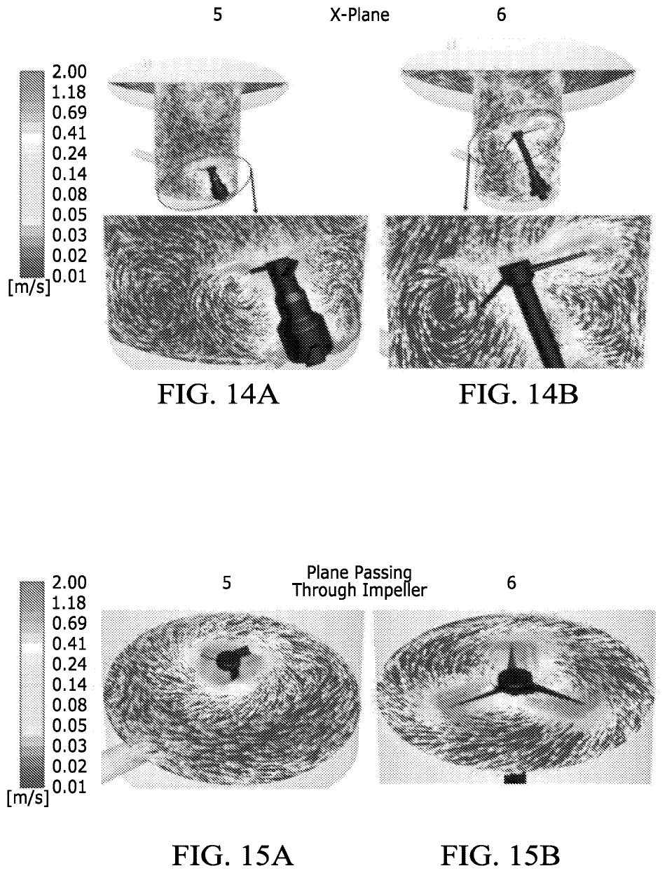

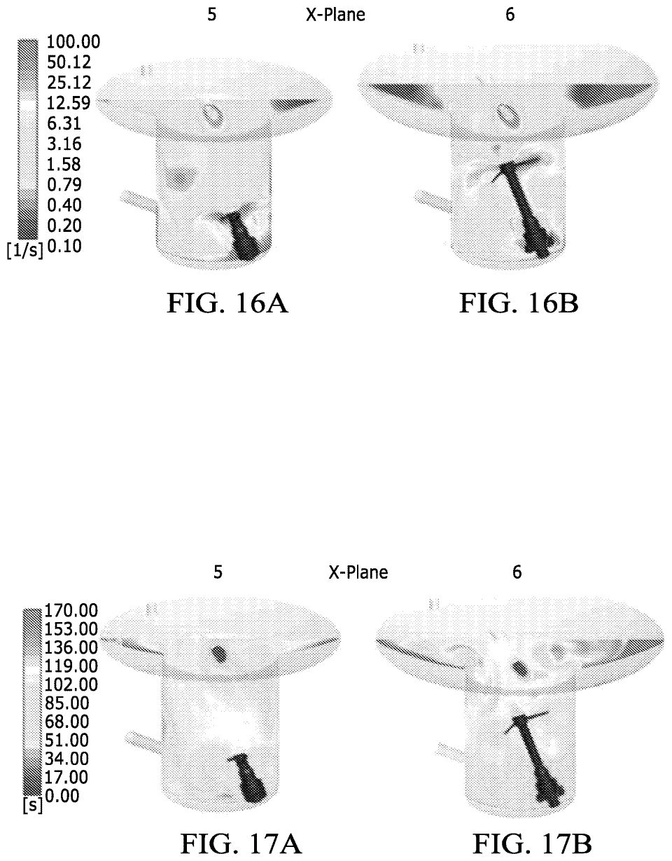

Flow field contours on the X-plane for the MC2 cases are shown in FIGS. 12A and 12B and the horizontal plane passing through impeller for the MC2 cases are shown in FIGS. 13A and 13B. Due to the higher agitation rate, the flow field contours for the 4 inch impeller show a high velocity near the impeller region than the 10 inch impeller. However, the 4 inch impeller also has a few low velocity zones in the stem portion 102 of the tank 100, which indicates a low region of influence for the 4 inch impeller. FIGS. 14A-15B show velocity vectors on the X plane and the horizontal plane passing through impeller, respectively, for the MC2 cases.

The spatial distribution of shear rate on the X plane for the MC2 cases is shown in FIGS. 16A and 16B. By comparing the results in this section with the cases in MC1, the average shear rate for the tank with a 210 L working volume, the 10 inch impeller, and a 207 rpm agitation rate is comparable with the shear rate for the tank with a 210 L working volume, the 4 inch impeller, and a 345 rpm agitation rate.

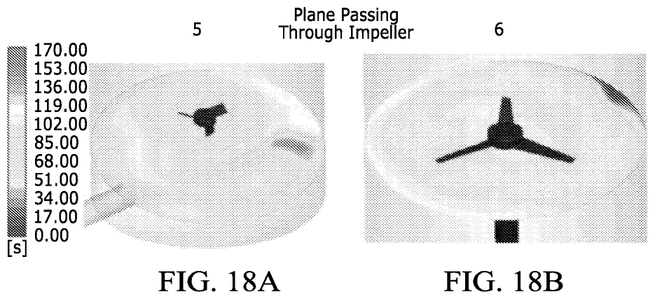

In FIGS. 17A-18B, the spatial distribution of mean age on the X-plane and the horizontal plane passing through impeller for the MC2 cases are shown. By comparing these results with the mean age results for MC1, it can be determined that a relatively better mixing performance can be observed for the 10 inch impeller, the 210 L working volume, and the 207 rpm agitation rate when compared to the 4 inch impeller, the 210 L working volume, and the 345 rpm agitation rate.

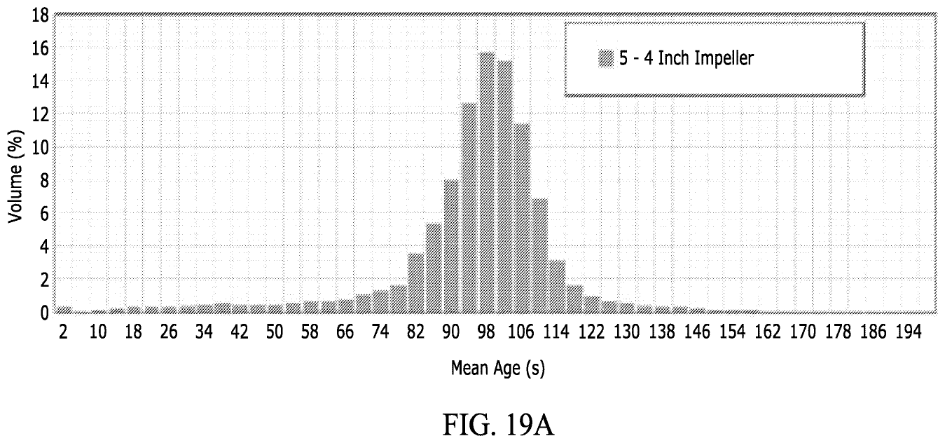

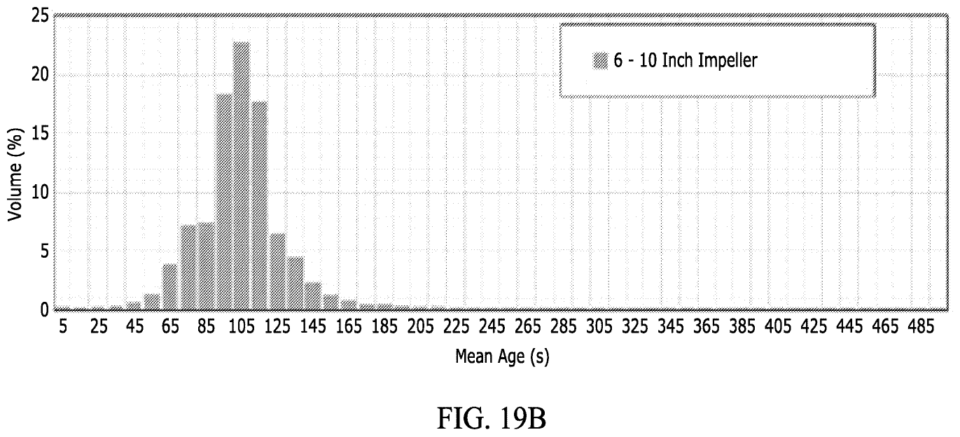

FIGS. 19A and 19B show a mean age histogram for the MC2 cases. The standard deviation of the mean age is given in Table 6 below. The 4 inch impeller, as shown in case 5, has a wider mean age distribution than the 10 inch impeller. For 10 inch impeller, as shown in case 6, ˜65% of the tank volume has a mean age close to the retention time (105 s). However, due to some low velocity regions near top surface of the fluid, the standard deviation for the 10 inch impeller is higher than the 4 inch impeller.

TABLE 6
Standard deviation of mean age for MC2 cases
Flow studies at nominal conditions

| | | | | | | | a_e | a_v | | | |
|---|---|---|---|---|---|---|---|---|---|---|---|
| | Working | Impeller | | Flow Rates (lpm) | | | Area | Volume | tau | | Sigma_e |
| | Volume | Speed | Diameter | Feed | Return | Flux | Weighted | Weighted | Retention | Sigma_e² | Standard |
| Case | (L) | (rpm) | (inches) | D2 | F2 | F1 | Ave. | Ave. | time (s) | Variance | Deviation |
| 5 | 140 | 345 | 4 | 96 | 88 | 8 | 89.86 | 97.08 | 87.5 | 1.16 | 1.077 |
| 6 | 210 | 207 | 10 | 120 | 110 | 10 | 105.37 | 122.5 | 105 | 1.33 | 1.151 |

A performance summary for the MC2 cases are shown in Table 7. It is observed that the 10 inch impeller has a good fluid pumping capacity with low power consumption. The 10 inch impeller shows more average velocity than the 4 inch impeller, with suitably low shear rates. Based on the modeling discussed herein, driving rotation of the 10 inch impeller at less than 250 rpm can maintain a volume average turbulence kinetic energy (TKE) of at least 0.005 m²/s² and/or have a turbulent dissipation rate (TDR) of at least 0.035 m²/s³.

TABLE 7
Performance Summary for MC2 Cases

| Parameters | Units | Case 5 | Case 6 |
|---|---|---|---|
| Impeller Tip Speed | m/s | 1.83 | 2.75 |
| Maximum Shear Rate | s⁻¹ | 9882.4 | 29907.6 |
| Impeller Power Consumption | kW | 0.0106 | 0.0098 |
| Impeller Pumping Capacity | m³/s | 0.008 | 0.0193 |
| Impeller Power Number | — | 4.78 | 0.22 |
| Impeller Flow Number | — | 1.31 | 0.34 |

Average Quantities

| | | | |
|---|---|---|---|
| Total Power Consumption | kW | 0.0106 | 0.0098 |
| Power Per-Unit Volume | kW/m³ | 0.075 | 0.047 |
| Average Strain Rate | 1/s | 6.04 | 4.8 |
| Volume Average Velocity | m/s | 0.146 | 0.156 |
| Volume Average TKE | m²/s² | 0.0073 | 0.0058 |
| Volume Average TDR | m²/s³ | 0.060 | 0.035 |
| Micromixing time calculated based | s | 0.18 | 0.161 |
| on Average TDR (epsilon) | | | |

The results from the CFD models provide a comparison of 4 inch impeller and 10 inch impeller performance in different scenarios, such as agitation rates and working fluid volumes, through velocity field, shear rate distribution, and mean age contours. The main takeaways from the simulated operating condition in the two modeling cycles are summarized as follows:

For the cases considered in this study, at the same agitation rate and working volume, the 10 inch impeller system has higher average velocity, higher average shear rate (7.14 s⁻¹ vs 4.22 s⁻¹), and better mixing performance as compared to the 4 inch impeller. This is valid for both low and high viscosity media, as considered in these modeling cycles.

To reduce the average shear rate for the 10 inch impeller in a fixed working volume, a reduced agitation rate of 207 rpm can be used instead of 345 rpm. In this case, the mixing performance of the 10 inch impeller with a 207 rpm agitation rate is better than the 4 inch impeller with a 345 rpm agitation rate. Further, the average shear rates for the cases are comparable (4.80 s⁻¹ vs 4.22 s⁻¹). Finally, it is believed that the 29907.6 s⁻¹ maximum observed shear rate in the tank with the 210 L working volume and the 207 rpm agitation rate falls within acceptable levels that would not compromise the quality of the drug product being created.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

Although the systems, methods, and elements thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention. Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method for mixing a liquid composition, the method comprising:
providing a tank having an impeller with a 10 inch blade diameter disposed therein;
filling the tank with a liquid composition comprising etanercept and one or more buffers to a level within the tank to cover the impeller;
operating the impeller to mix the liquid composition to a homogeneous state while maintaining a maximum shear stress imparted on the mixture of fluids at 125,000 s⁻¹ or below.

2. The method of claim 1, wherein the maximum shear stress imparted on the liquid composition is less than 55,000 s⁻¹.

3. The method of claim 1, wherein the maximum shear stress imparted on the liquid composition is less than 30,000 s⁻¹.

4. The method of claim 1, further comprising operating the impeller to maintain an average shear rate of less than 8 s⁻¹ throughout the liquid composition.

5. The method of claim 1, wherein the tank comprises a tulip-shaped tank with a bottom stem portion and a top bulb portion; and operating the impeller comprises driving the impeller via a connection through the bottom stem portion of the tank.

6. The method of claim 1, wherein operating the impeller comprises driving rotation of the impeller at less than 250 rpm.

7. The method of claim 6, wherein operating the impeller comprises maintaining a volume average turbulence kinetic energy of at least 0.005 m²/s².

8. The method of claim 6, wherein operating the impeller comprises having a turbulent dissipation rate of at least 0.035 m²/s³.

9. The method of claim 1, wherein operating the impeller produces a power/flow number ratio less than 1.

10. The method of claim 9, wherein operating the impeller produces a power/flow number ratio of less than 0.7.

11. The method of claim 1, wherein operating the impeller comprises driving rotation of the impeller to pull the liquid composition upward from a bottom of the tank.

12. The method of claim 1, further comprising performing a buffer exchange pursuant to an ultrafiltration/diafiltration process.

13. The method of claim 1, wherein filling the tank with the liquid composition comprises filling the tank with a concentrated therapeutic product including etanercept and a final buffer.

14. The method of claim 1, wherein the tank has a volume of 210 L or less.

15. The method of claim 1, wherein the tank has a volume of 140 L or less.

16. The method of claim 1, wherein the impeller includes three blades having a tapering profile.

17. The method of claim 1, wherein the liquid composition in the homogenous state has a viscosity between about 3 cp and about 8 cp.
